(19) United States
(12) Patent Application Publication
(10) Pub. No.: US 2020/0094123 A1
(43) Pub. Date: Mar. 26, 2020

(54) Tracking Device Architecture

(71) Applicant: Sports Performance Tracking Pty. Ltd., Victoria (AU)

(72) Inventors: Henry Walshaw, Victoria (AU); William Strange, Victoria (AU); Matthew James Adams, Victoria (AU); Daniel Berton, Victoria (AU)

(21) Appl. No.: 16/619,588

(22) PCT Filed: Jun. 6, 2018
(86) PCT No.: PCT/AU2018/050562

§ 371 Date: Dec. 5, 2019

(30) Foreign Application Priority Data
Jun. 6, 2017 (AU) 2017902150

(51) Int. Cl. A63B 71/06; G16H 20/30; G16H 40/67; G01S 19/47

(52) U.S. Cl. CPC A63B 71/0622; G16H 20/30; A63B 2220/12; G01S 19/47; A63B 2225/50; G16H 40/67

(57) ABSTRACT

The present invention is concerned with a tracking device (10) including: a sensor system (12) configured to determine at least one movement parameter of the tracking device and output movement parameter data; a power supply (34) arranged to supply electrical power to the tracking device; a memory (30); and a processor system (24) including: a data processor (26) configured, in accordance with one or more operational parameters stored in the memory, to: control the determination of said at least one movement parameter by the sensor system; and process the movement parameter data and output one or more derived parameters; and a second data processor (26) configured to: operate independently from the data processor; and manage access to the memory and storage therein of: the one or more operational parameters; a log of said movement parameter data output from the sensor system; and said derived parameters.

### FIELD OF THE INVENTION

The present invention relates to the hardware architecture and operation of tracking devices. Illustrative embodiments will be described in connection with tracking devices used to track people, typically sports people during a sporting activity, however, it should be understood that embodiments of the present invention may be used in tracking devices applicable to other types of tracking.

### BACKGROUND OF THE INVENTION

Tracking devices are known to be used for tracking the movement of a carrier of the device. Examples of tracking devices may be found in US 2016/0253471 and US 2016/0350100 and typically include:
A global navigation satellite system (GNSS) receiver to determine the location of the tracking device.
Optionally, a sensor system e.g. force and/or acceleration sensor system to determine instantaneous motion or orientation of the tracking device.
A communications interface, such as a USB or Wifi interface for connecting to an outside system.
A memory for storing logged data from the GNSS receiver and other optional secondary sensors.
A control system for controlling operation of the sensors and data gathering as well as communications with outside systems.
A power source, typically a battery, for powering the device in use.

Because such tracking devices typically need to be carried by a person or attached to their equipment, it is highly advantageous that they are as small and light as possible in order to be easy to carry and unobtrusive. Accordingly, the systems tend to be simple as possible to achieve long battery life with a small size. However, with these limitations come trade-offs in system functionality.

For example, such systems tend to be “set and forget” in ordinary day-to-day use, in that systems are largely unable to be easily updated, but instead need a full firmware update to make system changes. This has limitations in the practical use of these devices, as firmware updates can be costly and are potentially fragile (a failed firmware update can render a device inoperable) and are typically difficult to reverse. Firmware updates require a full reset of the device and thus require erasing any stored sensor data. Firmware updates may also result in the resetting of fundamental data of the device such as a MAC address, which may have implications for the registration of a device with associated systems (if the MAC address is used to identify the particular tracking device relative to others in the system).

Furthermore, updating the firmware on a device may be time consuming. This drawback will be magnified in team situations, where each member of the team possesses their own tracking device, and the firmware update needs to be provided to all of the devices.

Accordingly, the present invention seeks to provide an alternative architecture and method of use of a position tracking device.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

According to a first aspect of the present invention there is provided a tracking device including:

a sensor system configured to determine at least one movement parameter of the tracking device and output movement parameter data;

a power supply arranged to supply electrical power to the tracking device;

a memory; and

a processor system including:
a data processor configured, in accordance with one or more operational parameters stored in the memory, to:
   control the determination of said at least one movement parameter by the sensor system; and
   process the movement parameter data and output one or more derived parameters; and

a second data processor configured to:
operate independently from the data processor; and
manage access to the memory and storage therein of: the one or more operational parameters; a log of said movement parameter data output from the sensor system; and said derived parameters.

A tracking device according to the present invention includes first and second processors that operate independently of each other, or in other words according to a master-master setup. Such architecture conveniently allows the functions of the tracking to be conveniently divided with each processor allocated tasks to which they are best suited. In particular, the data processor is responsible for controlling the primary tracking functionality of the tracking device (including undertaking floating point operations to determine derived parameters) and overall system control, with the second data processor being utilised as a dedicated memory manager that brokers access to the memory and to any connected communications interface.

Typically, the data processor and second data processor operate independently through and communicate with each other via an asynchronous communication channel. Utilising non-homogeneous (or in other words ‘heterogeneous’) processors allows for the dual, independent-processor architecture of the present invention to be further leveraged, in that processor capabilities and power requirements can be tailored and optimised to the specific tasks that the individual processors are to perform.

Preferably, the sensor system includes:

a global navigation satellite system (GNSS) receiver to determine a location of the tracking device; and

a secondary sensor system to determine a kinetic parameter of the tracking device.

The secondary sensor system can include one or more sensors selected from a list comprising: an accelerometer, a magnetometer and a gyroscope.

Optimally, the GNSS receiver is communicatively coupled to the first processor via an asynchronous communication channel; and the secondary sensor system is communicatively coupled to the first processor via a synchronous communication channel. Coupling the processors and communications channels in this way locates real time communication channels in closer proximity to the processors leading to more efficient calculation of the derived parameters. At the same time, the bulk transfer interface is located in closer proximity to the processor with the superior bulk transfer capabilities.

The operational parameters may include one or more of a kinetic parameter sampling rate, kinetic parameter filtering parameter, location data sampling rate and a movement parameter data processing algorithm and parameters therefor.

Preferably, the second data processor is configured to store updated operational parameters in the memory in response to a received input.

Typically, the tracking device according to the invention further includes a communications system configured to communicate with an external device or system. According to preferred embodiments, the communications system includes first and second distinct communications interfaces. One of the communications interfaces may be a wireless interface arranged under the control of the data processor to communicate during a session of the tracking device, data representing the derived parameters received.

Optionally, the second data processor and memory are communicatively coupled via a synchronous communication channel.

According to preferred embodiments, the second data processor is configured to selectively store data in the memory in response to the rate of receipt or volume of movement parameter data received from the sensor system.

According to an alternative embodiment, the first communications interface can be arranged to communicate data representing derived parameters, and the second communications interface can be arranged to communicate data representing said operational parameters for use by the first processor. One or both of the second communications interfaces can be used to communicate data representing the log. However, in a preferred form the second interface is used.

Said plurality of communications interfaces can communicate via different communications channels. For example a first communications interface can be a wireless communications interface, for example, Bluetooth low energy. The second communications interface can be a wired communications interface, for example USB.

The first communications interface is preferably controlled by the first processor.

The second communications interface is preferably controlled by the second processor.

Said log can further include derived parameter data.

The movement parameter data representing said at least one movement parameter can be raw movement parameter data or processed data representing said movement parameter data.

In one form the tracking device of the first aspect of the present invention can be adapted to perform a method according to an embodiment of the second or third aspects of the invention as set out below.

In a second aspect there is provided a method of operating a tracking device of the type including, a sensor system; a memory; a processor system; and a power supply; said method including:

during a session, determining at least one movement parameter of the tracking device using the sensing system;

storing a log of said movement parameter data representing said determined movement parameter(s) in the memory;

processing movement parameter data representing said determined movement parameter(s) to output derived parameter data; and

transmitting the derived parameters to a remote device during the session.

The method can include transmitting the log including movement parameter data representing said determined movement parameter(s) to a remote device. Preferably the log of said movement device is transmitted after the conclusion of the session. Said log preferably includes the movement parameter data from which the derived parameter data is generated. The log may also include derived parameter data.

The method can further include, one or both of:

determining at least one movement parameter of the tracking device on the basis of configuration data stored in said memory; and

processing movement parameter data on the basis of configuration data stored in said memory.

The method can include updating the configuration data to change the manner in which either or both of said movement parameter or derived data is generated.

In a third aspect there is provided a method of operating a tracking device of the type including, a sensor system; a memory; a processor system; and a power supply; said method including:
(a) during a session, determining at least one movement parameter of the tracking device using the sensing system; and optionally processing movement parameter data representing said determined movement parameter to output derived parameter data, wherein either or both of the determining at least one movement parameter or processing of the movement parameter data is performed in accordance with configuration data;
(b) storing a log including movement parameter data representing said determined movement parameter in the memory;

said method further including:

receiving updated configuration data and in a subsequent session performing step (a) using the updated configuration data.

The method can include during a session, transmitting the derived parameters relating to at least part of said session to a remote device.

The method can include transmitting the log to a remote device. Preferably the log is transmitted after the conclusion of a session.

The log may also include derived parameter data.

Said log preferably includes the movement parameter data from which the derived parameter data is generated.

Preferably the tracking device includes a processor system including:

a first data processor configured, in accordance with the one or more operational parameters, to control the determination of said at least one movement parameter by the sensor system; and/or process movement parameter data and output one or more derived parameters; and

a second processor configured to update the operational parameters of the first processor in accordance with a received updated configuration data.

In the above aspects it is preferable that the tracking device is a sports tracking device. The sports tracking device can have a form factor which adapts the tracking device for carriage by the sports person on their clothing or equipment.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a schematic diagram of the architecture of a tracking device according to one embodiment.

FIG. 2 is a flowchart illustrating a process for logging movement parameter data using the device of FIG. 1.

FIG. 2B illustrates a further embodiment of a process for logging movement parameter data and derived parameter data using s dual processing architecture.

FIG. 3 is a flowchart illustrating a process for transmitting the log to a remote device.

FIG. 4 is a flowchart illustrating a process for transmitting derived parameter data to a remote device using the device of FIG. 1.

FIG. 5 is a flowchart illustrating a process for transmitting the log to a remote device.

FIG. 6 is a flowchart illustrating a process for updating configuration data.

### DETAILED DESCRIPTION OF THE EMBODIMENTS

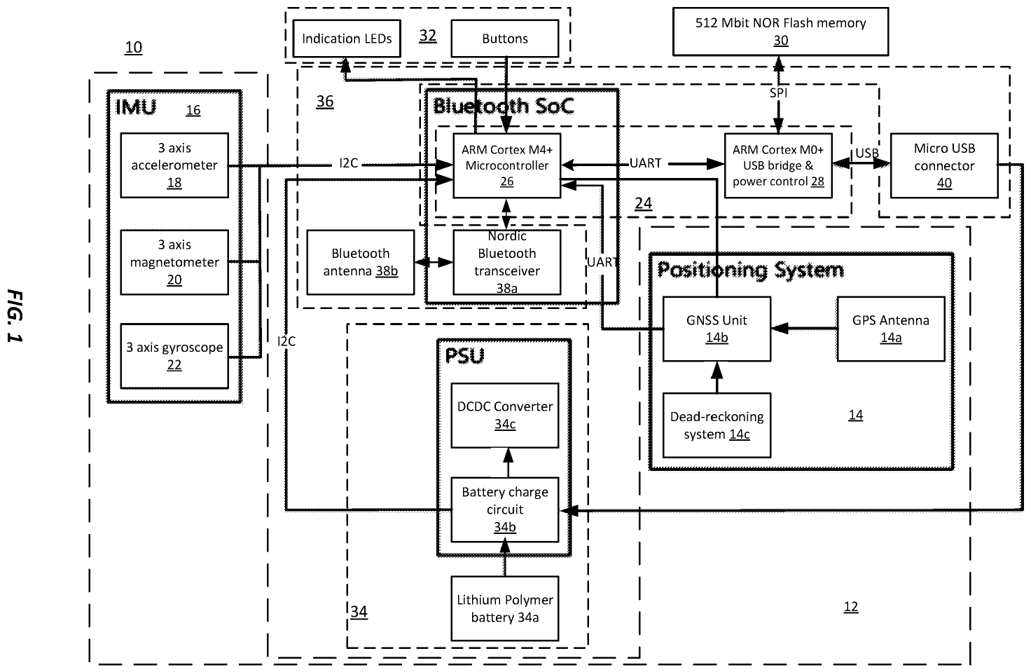

FIG. 1 is a schematic diagram illustrating the architecture of a tracking device according to an embodiment of the present disclosure. The tracking device 10 may be a sports tracking device or other unit which may be mounted to an entity to be tracked. By way of overview, the way of tracking device 10 includes the following component subsystems which work together to enable tracking of the device:
Sensor system 12—The sensor system 12 is configured to determine a value of a movement parameter of the tracking device 10. In this example, the sensor system 12 comprises a global navigation satellite system (GNSS) 14 and a secondary sensor system 16. The secondary sensor system in this case includes a three axis accelerometer 18, a three axis magnetometer 20 and a three axis gyroscope 22. The GNSS 14 includes an antenna 14a for receiving signals from satellites of a constellation of GNSS satellites, such as GPS, Galileo or Beidou satellites. The associated GNSS processor unit 14b processes the receiver data and outputs a determined location. The GNSS processor unit 14b can additionally include a dead reckoning system 14c for use in the event of impaired satellite connectivity or other situations where it may be necessary to interpolate between measured locations. The secondary sensor system 16 outputs a series of measurements corresponding to sensor type—i.e. the accelerometer 18 outputs acceleration measurement vectors, the magnetometer 20 outputs direction measurement vectors, and the gyroscope 22 outputs measurements of rotational motion.
Processor system 24—The processor system 24, includes a first data processor 26 and a second data processor 28. The first data processor 26 controls the primary tracking functionality of the tracking device 10, including the determination of derived parameters and overall control of the operation of the sensor system 12. The first data processor 26 is capable of performing floating point calculations in order to efficiently calculate derived parameters. In the present example, the first processor 26 can be an ARM, Cortex M4+ processor. The second processor 28 controls the data storage control including storage of movement parameter and derived parameter data as well as configuration data used by the first data processor 26 in performing its operations. Accordingly there is no need for the second processor 28 to perform complex calculations, so a less sophisticated (lower cost, and lower power) processor can be used. In this example an ARM, Cortex M0+ processor is selected. Further details of the roles of each of the first and second data processors will become apparent upon reading the following description. It should be understood that the first and second data processors are described as incorporating being single microprocessors, however the invention should not be considered to be limited to this embodiment. For example a multiprocessor architecture could be used to perform the function of the first or second data processors described herein. Moreover, one or both of the data processors could be implemented using different data processing system, such as a field programmable gate array (FPGA) or application specific integrated circuit (ASIC). However it is advantageous for at least some aspects of the present invention that the first data processor 26 operates in accordance with re-programmable configuration data.
Memory 30—Memory 30 is used to store all recorded data and configuration data for the tracking device. Access to the memory 30 is controlled by the second processor 26. The memory can include one or more memory chips or modules. The memory 30 can be arranged into memory address blocks dedicated to certain purposes e.g. storage of configuration parameters, storage of log data being the two of primary concern here. The memory can also have a reserved, secure region that is not accessible by the user or erasable during firmware update. The use of memory 30 to store configuration data for the first data processor 26 facilitates the ability for the second data processing system 28 to manage updates of the configuration data used by the first data processing system 26 during its operation in a manner that will be described below.
User interface 32—The user interface 32 can include one or more input and/or output devices such as buttons or indication LEDs. As would be appreciated by those skilled in the art, it may not be necessary to have sophisticated user interactions on a simple tracking device. In fact, it may be sufficient to have a single push button through which a user can turn the device on and off, and possibly cycle through operational modes; and a single LED which can be selectively illuminated to provide feedback to a user as to the current status of the tracking device 10. However, in other embodiments a more sophisticated interface, such as a display screen, possibly a touch screen display, may be used to provide additional information to a user and receive complex inputs.
Power supply unit 34—The power supply unit 34, includes a battery 34a, such as a lithium polymer battery. Power is supplied to the battery 34a via a power connection, which in this case comes from a circuit connected to a power pin of a USB connector. The power which is delivered to the power supply unit 34 is converted to an appropriate voltage by DC-DC converter 34c and charge is delivered to the lithium polymer battery 34a in accordance with a battery charge management scheme implemented by the battery charging circuit 34b. The battery charging circuit 34b also manages the power supply to the remainder of the tracking device 12 in order to protect the battery 34a from incorrect charging and discharging.
Communications system 36—The communications system 36 handles communication with remote devices. As would be appreciated, the remote devices can comprise any type of external computing or data processing or storage device. However, most commonly the remote device will be a personal computer or mobile computing device such as a tablet computer or smart phone. In the present embodiment, the communication system includes two external communication interfaces, namely a first communications interface 38, which operates wirelessly, and a second communications interface 40 which uses a wired communications channel. The first communications channel preferably operates according to one of the following communications protocols, Bluetooth, Bluetooth low energy, zigbee, wifi, NFC, Nike+, IrDA, or the like. The second communications interface 40 preferably operates according to one of the following communications protocols RS242, RS485, USB, Ethernet, or the like. In this example, the first communications interface 38 is operated under control of the first data processor 26 and the second communications interface 40 is controlled by the second data processor 28. As will become apparent from the following description, this enables a convenient transmission of certain data during the use of the tracking device under control of the first data processor 26, and also robust communication of larger amounts of data via the second communications interface 40 under control of the second data processor 28.

In the present example, the second communication interface 40 has a micro USB connection via which a communication with a USB host can take place, and over which data can be supplied to the tracking device. The first communications interface 38 is in this example, a Bluetooth low energy interface and includes a Bluetooth transceiver system 38a coupled to a Bluetooth antenna 38b which operates in a manner which will be known to those skilled in the art.

As indicated in FIG. 1, data and electrical connections are maintained between each of the systems described above. It will be apparent to those skilled in the art that it will be appropriate to use different forms of connection between different system systems, for example, connection between the battery charge circuit 34b and the secondary sensor system 16 and the first data processor 26 can be via I²C whereas connection between the GNSS system 14 and the first processor 26 can be via UART connection. Similarly, connection between the two processors 26 and 28 can also use UART communications between them. A USB connection would be provided between the micro USB connector and the second processor 28. The power supply line derived from this connection will run to the power supply unit 34. Memory connection can be via any peripheral interface mechanism, but in this case a serial peripheral interface bus (SPI) is selected.

Using an architecture such as that described above and illustrated in FIG. 1 enables the functions of the tracking device to be conveniently divided, which may improve the ability to perform system updates and output data for use midway through a session. In the preferred embodiment, the system architecture is setup so that the first processor performs all complex calculations and system control functions, such as controlling the collection of movement parameters from the sensing system. Calculation of any derived parameters are also performed by the first data processor 26. Also, the first data processor 26 also controls on board user interface functions.

The second data processor 28 has, as its primary functions:
(a) memory control e.g. reading and writing movement parameter data and derived parameter data to the memory 30, storage of configuration data on the memory 30.
(b) communication with external, remote devices to either:

send the stored log data to the external devices e.g. for analysis, visualisation or other data processing, and

receive configuration data which is used to control the operation of the first data processor 26.

Generally speaking, the configuration data will be one or more operating parameters which are used by the first processing device 26 to control the operation of one or other of the components of the device, or perform data processing on the recorded movement parameters. For example, configuration data could include sampling rates for the sensors comprising the secondary sensor system 16 or the rate at which the location data should be recorded from the GNSS system 14. It may also include data processing algorithms, or parameters therefore, for use in calculating derived parameters from the recorded movement parameters.

The second data processor 28 has the role of performing all reads and writes to the memory 30 as well as assembly of data for storage in memory 30. This is advantageous as the memory management can be handled in parallel with the first data processor's 26 tasks thereby increasing the resultant throughput on the device in total. Moreover it can be advantageous in terms of power usage, because the lower power processor (second data processor 28) carries out more data transfer tasks.

A tracking device will be used to track movement of a carrier in periods called sessions. A session typically begins when a device is activated and ends when the device is turned off. However other start or end events may be used e.g. a user input received via the user interface 32 or via a communication interface, to demarcate the start and end of a session.

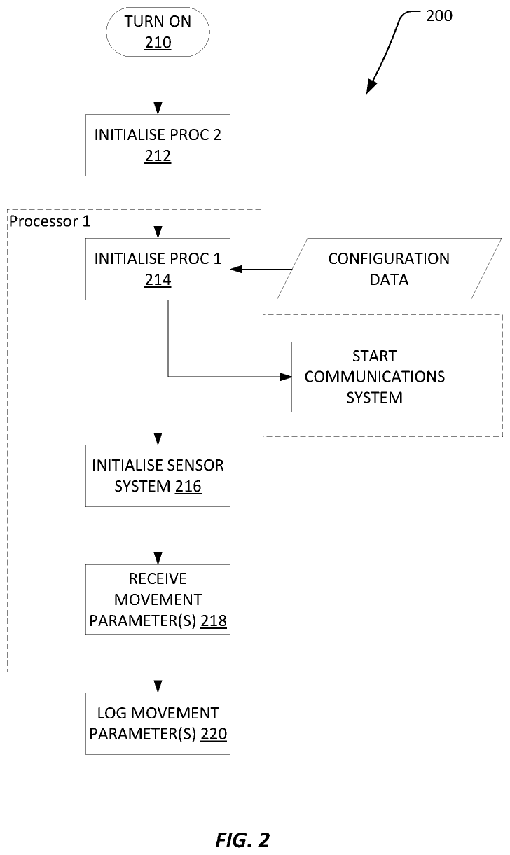

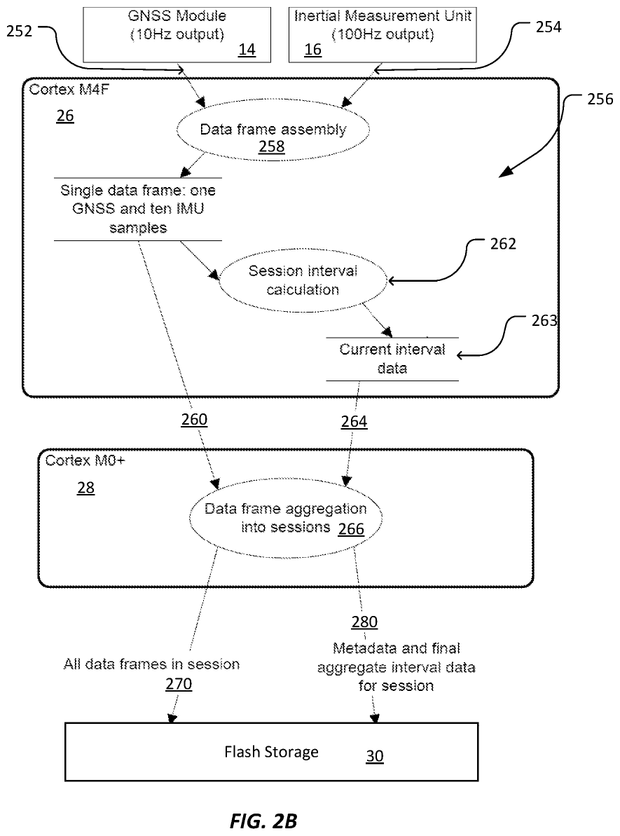

FIG. 2 illustrates a process 200 for turning on a tracking device of the type shown in FIG. 1, which may be used to start a session. The process begins by turning the device on at 210. This causes initialisation 212 of the second processor 28. The second processor 28 triggers initialisation 214 of first processor 26. During initialisation the second processor reads configuration data from the memory 30. \

Once initialised the first processor begins initialisation 216 of the sensor system 14, which causes the GNSS system to begin acquiring signals from satellites, perform any calibration, or diagnostic checks on the sensor systems etc. Once initialised the sensor system 14 begin generating movement parameters for the tracking tag 218, which are then logged 220 in the memory 30. The generation and/or logging is performed in accordance with the configuration data used to initialise the first processor. In one form this can include setting sensor sample rates, or filtering parameters. It could also or alternatively include selectively storing data according to the configuration data if sufficient control over the rate of movement parameter data generation by the sensor system is not possible. The process of receiving movement parameter data from the sensor system 14, and storing thereof in a log in the memory 30 will be continued under control of the first processor 26 until the end of the session. An exemplary process is illustrated in FIG. 2B.

The process 250 begins by receiving movement parameter data from the GNSS sensing system 252 and secondary sensing system 254. The data from the GNSS system 14 is measured at a first rate (e.g. of 10 Hz) whereas the secondary sensor system generates measurements at a faster rate (e.g. 100 Hz). Other relative rates are possible.

Next 256 the first data processing system 26 assembles a sample frame 258 containing data from the sensor system 12 over a time interval.

Table 1 sets out an example sample frame containing movement parameter data stored in the memory 30 for each received GNSS position received from the sensor system 12. As noted, the GNSS system may operate at a lower sensing rate than a secondary sensor. In one example the GNSS 14 generates data points at rate of 10 Hz, but the sensors 18, 20, 22 of the secondary sensor system 16 operate at 100 Hz. Thus for each GNSS measurement, 10 measurements for each secondary sensor can be stored. In this example movement parameter data is stored in the memory 30 in a sample frame which collects data for the 100 ms period between GNSS measurements, and contains 10 secondary sensor values from each GNSS sample value.

TABLE 1
Data stored for each sample frame
Time in ms
LATUTUDE
LONGDITUDE
HEIGHT
SPEED
GPS quality data
COMPASS_DEG
ACCEL_X-10 sequential samples from accelerometer 18
ACCEL_Y-10 sequential samples from accelerometer 18
ACCEL_Z-10 sequential samples from accelerometer 18
GYRO_X-10 sequential samples from gyroscope 20
GYRO_Y-10 sequential samples from gyroscope 20
GYRO_Z-10 sequential samples from gyroscope 20
QUART_X-10 sequential values calculated based on
Magnetometer 22 output and gyroscope 20 output
QUART_Y-10 sequential values calculated based on
Magnetometer 22 output and gyroscope 20 output
QUART_Z-10 sequential values calculated based on
Magnetometer 22 output and gyroscope 20 output
QUART_W-10 sequential values calculated based on
Magnetometer 22 output and gyroscope 20 output
CHECKSUM

As will be appreciated the data making up a frame will differ from tracking device to tracking device and may not include all data, for example speed could be omitted, because it could be re-generated from the other stored movement parameter data. The log of data stored can include both movement parameter data and derived parameter data. In the example above, speed and the four quarternion values (QUART_X . . . QUART_W) are generated by calculation based on movement parameter data, and thus can be considered as derived parameter data. To avoid confusion the movement parameter data that represent a determined one movement parameter could be raw data derived from the sensor system 12 or processed data that represents the derived movement parameter. To give some examples the movement parameter data may be compressed, filtered (e.g. low or high pass filtered), normalised, truncated etc. prior to saving or processing to generate a derived parameter(s) value.

Returning to FIG. 2b, the sample frame, is then 260 passed to the second data processing system 28 for storage in the memory 30. In a preferred form the size of the sample frame advantageously is similar (and smaller than or equal to) the memory page size of the memory 30. This arrangement may allows more efficient memory usage and ultimately less memory usage than more wasteful formatting practices, and may assist in minimising memory requirements for the system 10 as a whole.

The first processor system 26 is also configured to process movement parameter data determined by the sensor system and output derived parameter data. This derived parameter data can comprise summary of the movement parameter data, for example:

an aggregate of certain movement parameter measurements (e.g. total distance travelled)

a value or statistical measure derived from the movement parameter measurements (e.g. an average speed)

a count of events detected in the movement parameter measurements (e.g. the number of rest periods, sprint efforts, impacts etc. detected);

or other value that can be calculated from the movement parameter data.

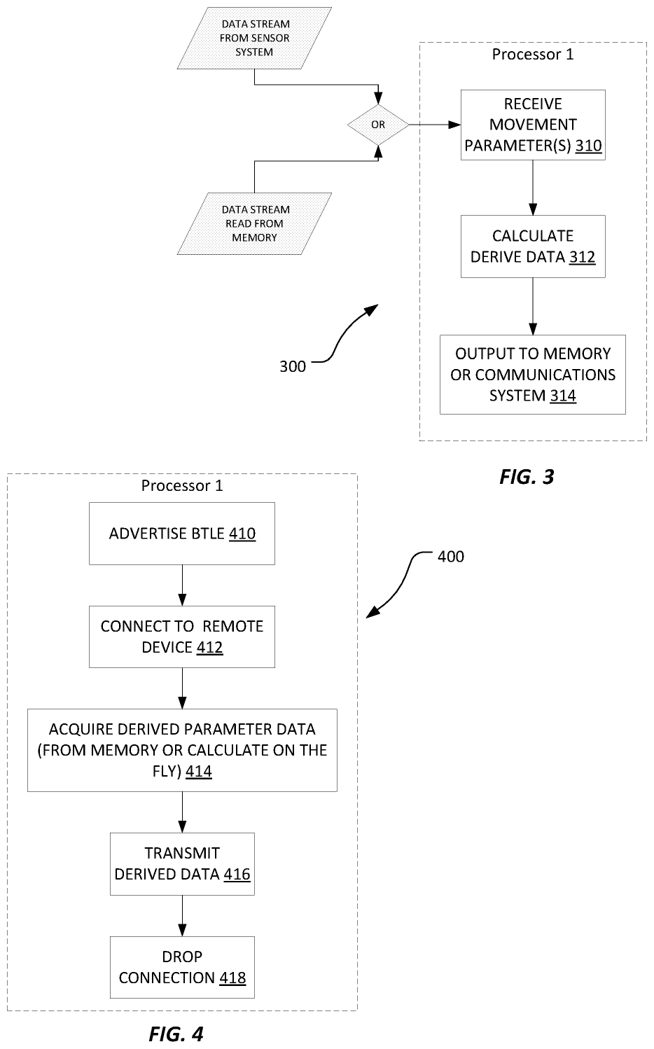

A process 300 for generating the derived parameter data is set out in FIG. 3. Importantly this process can take place during the current session (and as set out in FIG. 2B can be calculated on the fly). This enables the derived parameter data to be used as a snapshot of the current session while it is underway. The process begins by receiving 310 movement parameter data to be processed. The source from which this is received will depend on when the derived data is to be generated. In some embodiments the derived parameter data will be generated on the fly (as indicated at 262 in FIG. 2B), that is as the movement parameter data is generated. In this case the movement parameter data will be received from the sensor system (or related buffer) directly. In some embodiments the derived parameter data will be generated on-demand, or at set times (e.g. periodically or when certain events occur). In this case the movement parameter data will be read from the log stored in memory 30. Next 312 the first processor will process the movement parameter data to generate the derived parameter data. As indicated in FIG. 2B the derived parameter data will be passed 264 to the second processor system 28 for storage.

Two exemplary processes for generating a derived parameter, are set out below:

In a first example the derived parameter is a count of rests taken by a tracked player. This count will be generated from movement parameter data, in the form of speed and location data obtained from the GNSS system. The parameters of what constitutes a rest can be uploaded to the memory 30 as operational parameter data. In this example a player will be determined to have taken a rest if they have had a speed less than a threshold speed for a time greater than a rest threshold time.

At the start of a session, the player starts with a rest count of 0, and is assumed not to be at rest (at rest flag =false). For each valid GNSS reading of speed and location received from the sensor system 12 the processor 26 performs the following routine:

if speed lower than rest threshold speed:

if already at rest, increment the rest time counter by the sample interval

otherwise set the “at rest” flag to “true”

If speed not lower than rest threshold speed, set the rest flag to “false”, and if current rest time is greater than the rest time threshold, increment the rest count and reset the rest timer counter.

In a second example the tracking device can be configured to count impacts and return this data in a snapshot. In this example the movement parameter data being processed is generated by the accelerometer 18 and is processed in accordance with operational parameter data defining an impact acceleration threshold.

In this example, an acceleration magnitude is calculated from instantaneous acceleration reading (sample.x, sample.y, sample.z) by the formula:

(x acceI²+y acceI²+z acceI²)^{0.5}

Impacts are judged over a rolling 3 second analysis window.

For each acceleration reading in a stream the processor 26 calculates the acceleration magnitude, and add it to the window dropping the oldest value if the window is full.

In the current sample window, if the standard deviation of the accumulated acceleration magnitudes is greater than an impact threshold, then if not currently in an impact, set the impacting flag to true and increment the impact count by 1.

As discussed elsewhere herein, the parameters that define a rest or high force will depend on the activity being performed and the skill and fitness level of the person being tracked. Thus having an ability to readily change the operational parameters of the tracking device to adapt to a particular user, without pushing a firmware update, may be advantageous. In some embodiments other data and parameters used in the generation of a derived parameter data could be stored as operational parameters, for example any or more of, the size of the analysis window, calculation used to determine the current acceleration, and the measure used (e.g. standard deviation of the total force in the analysis window) to detect an impact.

The derived parameter data 263 will then either be stored for later use (see 280 in FIG. 2b) or passed to the communication system for transmission to a remote device. Table 2 illustrates a data packet structure that can be assembled by the second data processor system using the calculated derived parameter data generated by the first data processing system 26. This snapshot of the tracking session can be either stored or sent to a remote device. As can be seen from Table 2 the data structure includes:

Overhead/metadata fields including data used for management (fields 1-3, 14) and time period identification (fields 4-5); and

Derived data fields that carry derived parameter data (fields 6-13).

TABLE 2
Snapshot packet structure

| Field | No of bytes | Data |
|---|---|---|
| 1 | 4 | Device ID, manufacturing data |
| 2 | 10 | Device nickname |
| 3 | 6 | Session start time (millisecond) |
| 4 | 2 | Session length |
| 5 | 2 | Number of metres travelled within session |
| 6 | 2 | Number of metres travelled at or above |
| | | sprint threshold |
| 7 | 2 | Count of sprint periods |
| 8 | 2 | Max acceleration |
| 9 | 2 | Impact count |
| 10 | 2 | Total rest time |
| 11 | 2 | Number of rest periods |
| 12 | 12 | Other derived data |
| 13 | 4 | Check sum |

Returning to FIG. 2b, in a preferred form the second data processor system 28 continues to receive (260, 264) and store 266 the sample frames (table 1, 270) and snapshot data (table 2, 280) in the memory 30 until it receives a signal that the session has concluded.

FIG. 4 illustrates a method of transmitting the derived parameter data to a remote device during a session. The process 400 begins by forming a network connection between a communications system 36 of the tracking device 10 and a remote device. Because it is taking place mid-session it is highly advantageous that the connection is made via a wireless communications channel. In this example the connection channel is a Bluetooth low energy channel. The Bluetooth transceiver 38a operates in an advertising mode 410 to enable discovery by the remote device. Once discovered the remote device connects 412 to the tracking device 10. This is preferably performed using a secure handshake protocol. The first processor then acquires 414 the derived parameter data to be sent (e.g. by calculating it on the fly as described above or by reading it from memory 30. As set put above the derived data will typically be formatted into one or more data packets having a particular data format that can be used by the remote device, and transmitted 416 across the communications channel. Finally the connection is dropped 418. The dropping of the connection can occur in any known manner, e.g. the tracking device may drop the connection unilaterally once the derived data is transmitted; in response to receiving an acknowledgement signal from the remote device, or after a predetermined time period.

In a typical use case the transmission of the derived parameter data will occur whenever the tracking device comes within range of a remote device that it can connect to. For example, whenever a player wearing a tracking device leaves the field, e.g. at a break in play such as half time or the like; when a player is substituted from the field; when the player comes near to a remote device but remains in action e.g. a track cyclist passes by a start/finish line or a player comes near to a sideline receiver etc. At this time, without any user interaction, the derived parameter data is uploaded for use by the remote device and a user thereof—such as a coach or media user.

In an embodiment, the derived parameter data is a relatively small amount of data, e.g. less than 100 Bytes (only 64 bytes in the example above) so can be transmitted in a short amount of time. Moreover the first processor can continue logging movement parameter data during data transmission, so the session does not need to end.

Once a session is complete however, the full log of movement parameter data can be transferred to a remote device, e.g. to enable analysis of it. This is done by connecting the tracking device 10 to a remote device via the communications system 36. This can preferably take place over the second communications system 40.

It is worth noting that the remote device to which the derived data is transmitted during a session does not have to be the same remote device to which the log data is transmitted at the conclusion of the session. In fact, it is envisaged that they will be different devices in many use cases. For example it may be more convenient for a coach or trainer to use a handheld device, such as a phone or tablet computer to view the derived parameter data mid-session (e.g. if they are on a field or the sidelines), but for detailed analysis of post-session log data it may be more convenient to use a desktop computer.

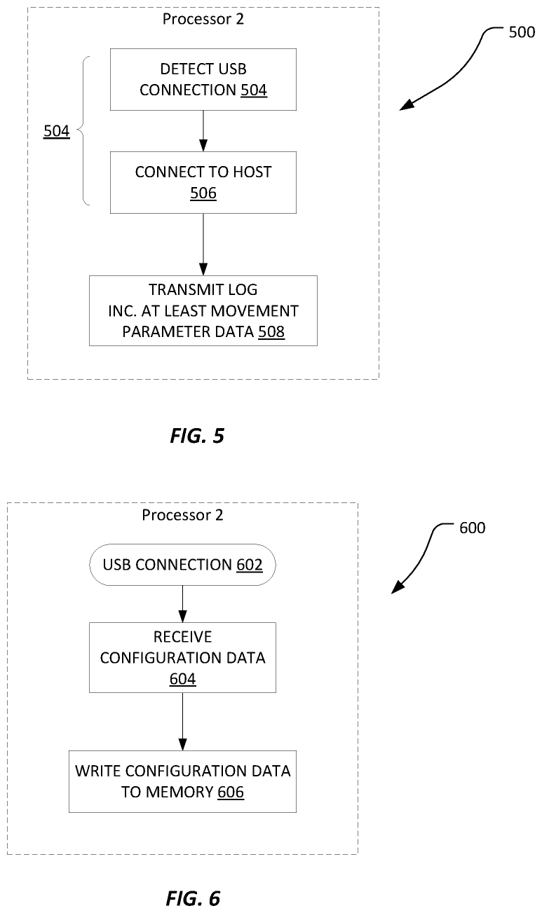

The upload of the log of movement parameter data can operate as set out in FIG. 5. The process 500 begins by making a network connection 502 using the second communication system 40. In the case of a USB connection the connection is detected 504 and a data connection is made with the host in step 506. Once the connection is established the second processor 28 can access the memory 30 and cause transmission of the log of movement parameter data to be transmitted 508 to the remote device. If the memory additionally stores derived parameter data, this may also be transmitted to the remote device. Any metadata associated with the movement parameter data and/or derived parameter data can also be transmitted.

As noted above the second communications interface 40 can additionally be used to receive configuration data for use by the first data processor 26. This process is performed by the second data processor using the second communications interface 40. This scheme enables a safe and convenient way to update the operation of the first processor 26, as it is no longer necessary to use a firmware update to change how the data processing system 24 operates. The process for updating the configuration data is set out in FIG. 6. The process 600 occurs after establishment of a data communication with a host system 602. The host system will typically be a personal computer running a software application or hardware driver configured to interoperate with the tracking device. If it is necessary to update the configuration data for the first data processor 26 the host pushes 604 a configuration data file to the tracking device. The second processor 28 causes the configuration data to be written 606 to a memory location in memory 30 where the first processor will access to acquire the relevant configuration data. The configuration data could be any one or more of:

Physical control parameters to adjust the operation of a hardware component of the tracking device;

Algorithms for processing movement parameters to generate derived data;

Parameters used in an algorithm used to generate derived data.

Configuration parameters can also relate to other operational data such as:

Battery management parameters, such as a level limit to warn as “low battery”

Human readable device identifier (nickname)

Security data such as a handshake key

Accordingly the present disclosure provides a method of operating a tracking device that enables changing configuration data between sessions to adjust the operation of the tracking device. This can have advantages in certain situations including, but not limited to:
(a) Where a single tracking device is being used to track the movement of different people at different times, and it would be advantageous to use different parameters for each person.
(b) Where a single tracking device is being to track movement in different contexts at different times (e.g. different sports, different conditions (indoor vs. outdoor)), and it would be advantageous to use different parameters for each person.
(c) Where over time the skill, competence, activity or fitness level of a person being tracked changes and different monitoring would be beneficial (e.g. a sports person returns to full fitness after injury).
(d) Where different derived parameter algorithms become available.

The dual processor architecture enables relatively easy updating compared to firmware updates, and because the full operating firmware of the first processor is not re-written, the chance of rendering it inoperably by an update is minimised.

Firmware updates can also become more simple with the architecture disclosed herein because the process can be controlled by a second processor, which is not being updated.

It will be understood that the invention disclosed and defined in this specification extends to all alternative combinations of two or more of the individual features mentioned or evident from the text or drawings. All of these different combinations constitute various alternative aspects of the invention.

1. A tracking device including:
a sensor system configured to determine at least one movement parameter of the tracking device and output movement parameter data;
a power supply arranged to supply electrical power to the tracking device;
a memory; and
a processor system including:
a data processor configured, in accordance with one or more operational parameters stored in the memory, to:
control the determination of said at least one movement parameter by the sensor system; and
process the movement parameter data and output one or more derived parameters; and
a second data processor configured to:
operate independently from the data processor; and
manage access to the memory and storage therein of: the one or more operational parameters; a log of said movement parameter data output from the sensor system; and said derived parameters.

2. A tracking device according to claim 1, wherein the data processor and second data processor are communicatively coupled via an asynchronous communication channel.

3. A tracking device according to claim 1, wherein the data processor and second data processor are non-homogenous.

4. A tracking device according to claim 1, wherein the sensor system includes:
a global navigation satellite system (GNSS) receiver to determine a location of the tracking device; and
a secondary sensor system to determine a kinetic parameter of the tracking device.

5. A tracking device according to claim 4, wherein:
the GNSS receiver is communicatively coupled to the first processor via an asynchronous communication channel; and
the secondary sensor system is communicatively coupled to the first processor via a synchronous communication channel.

6. A tracking device according to claim 4, wherein the operational parameters include one or more of a kinetic parameter sampling rate, kinetic parameter filtering parameter, location data sampling rate and a movement parameter data processing algorithm and parameters therefor.

7. A tracking device according to claim 1, wherein the second data processor is configured to store updated operational parameters in the memory in response to a received input.

8. A tracking device according to claim 1, further including a communications system configured to communicate with an external device or system.

9. A tracking device according to claim 8, wherein the communications system includes first and second distinct communications interfaces.

10. A tracking device according to claim 9, wherein one of the communications interfaces is a wireless interface arranged under the control of the data processor to communicate during a session of the tracking device data representing the derived parameters.

11. A tracking device according to claim 1, wherein the second data processor and memory are communicatively coupled via a synchronous communication channel.

12. A tracking device according to claim 1, wherein the second data processor is configured to selectively store data in the memory in response to the rate of receipt or volume of movement parameter data received from the sensor system.

13. A method of operating a tracking device of the type including, a sensor system; a memory; a processor system; and a power supply; said method including:
during a session, determining at least one movement parameter of the tracking device using the sensing system and outputting movement parameter data;
storing a log of said movement parameter data in the memory;
processing movement parameter data determined by the sensor system to output derived parameter data; and
transmitting the derived parameters to a remote device during the session.

14. A method according to claim 13, further including the step of transmitting the log including movement parameter data representing said determined movement parameter(s) to a remote device.

15. A method according to claim 14, wherein the log of said movement device is transmitted after the conclusion of the session and preferably includes the movement parameter data from which the derived parameter data is generated and derived parameter data.

16. A method according to claim 13, further including the step of determining at least one movement parameter of the tracking device on the basis of configuration data stored in said memory; and

17. A method according to claim 13, further including the step of processing movement parameter data on the basis of configuration data stored in said memory.

18. A method according to claim 13, further including the step of updating the configuration data to change the manner in which either or both of said movement parameter or derived data is generated.

19. A method of operating a tracking device of the type including, a sensor system; a memory; a processor system; and a power supply; said method including:
during a session, determining at least one movement parameter of the tracking device using the sensing system and outputting movement parameter data; and optionally processing said movement parameter data to output derived parameter data, wherein either or both of the determining at least one movement parameter or processing of the movement parameter data to generate derived parameter data is performed in accordance with configuration data;
storing a log of at least said movement parameter data in the memory;
said method further including:
receiving updated configuration data and in a subsequent session performing the determining step using the updated configuration data.

20. A method according to claim 19, further including the step of, during a session, transmitting the derived parameters relating to at least part of said session to a remote device.

21. A method according to claim 19, further including the step of transmitting the log to a remote device, preferably after the conclusion of a session.

22. A method according to claim 19, wherein the log also includes derived parameter data and/or the movement parameter data from which the derived parameter data is generated.

23. A method according to claim 19, wherein the tracking device includes a processor system including:
a first data processor configured, in accordance with the one or more operational parameters, to control the determination of said at least one movement parameter by the sensor system; and/or process movement parameter data and output one or more derived parameters; and
a second processor configured to update the operational parameters of the first processor in accordance with a received updated configuration data.
